(19) United States
(12) Patent Application Publication
(10) Pub. No.: US 2007/0088280 A1
(43) Pub. Date: Apr. 19, 2007

(54) Retention element for securing a catheter

(76) Inventor: Matthew A. Gomez, Bedford, TX (US)

Correspondence Address: LAW OFFICES OF ERIC KARICH, 2807 ST. MARK DR., MANSFIELD, TX, 76063, US

(21) Appl. No.: 11/522,703

(22) Filed: Sep. 18, 2006

Related U.S. Application Data
(63) Continuation-in-part of application No. 11/018,903, filed on Dec. 21, 2004.

(51) Int. Cl. A61M 5/32

(52) U.S. Cl. 604/174; 604/540

(57) ABSTRACT

A retention element has an axial portion having an internal conduit adapted to receive and engage a catheter. The retention element further includes a radially extending flange portion having an annular sidewall extending outwardly from the axial portion to an outer perimeter. The radially extending flange portion is adapted to contact the patient and secure the catheter, thereby retaining an inflatable balloon of the catheter in a position to seal the stomal tunnel. The radially extending flange portion is preferably generally conical in shape, such that only the outer perimeter of the radially extending flange portion contacts the patient at a contact location that is a distance from the stomal tunnel, and the annular sidewall does not contact the patient adjacent the stomal tunnel.

### CROSS-REFERENCE TO RELATED APPLICATIONS

This application for a utility patent is a continuation-in-part of a previously filed utility patent, still pending, having the application Ser. No. 11/018,903, filed Dec. 21, 2004.

### STATEMENT REGARDING FEDERALLY SPONSORED RESEARCH

Not Applicable

### BACKGROUND OF THE INVENTION

1. Field of the Invention

This invention relates to a device for retaining a catheter, and particularly relates to a supra pubic catheter with a moveable device along a catheter for improved sealing characteristics as well as the use of the system and method relating thereto.

2. Description of Related Art

Whether due to disease, stroke or Alzheimer's, a percentage of the population cannot urinate through their urethras. Such patients may be catheterized with a urinary catheter where the catheter is passed through the urethral of the patient until a distal end of the catheter is located in the patient's bladder. Thereafter, an expandable balloon is expanded to lodge the distal end of the catheter in the bladder. During catheterization, with a urinary catheter, urine drains from the bladder through the catheter, and through a drainage tube attached to a proximal end of the catheter to a drainage bag for collection therein.

Another system comprises the use of supra pubic catheters where the catheter passes through the abdomen wall of the patient until the distal end of the catheter is located in the bladder. This technique bypasses the urethra all together, and the bladder is connected to the outside of the body in the pelvic region. A surgical procedure is conducted where a stoma tunnel made of body tissue leads from the bladder to the outside of the abdomen.

Such supra pubic catheters have been used to drain urine by placing a urethral catheter through the tunnel or stoma. A balloon at a distal end is inflated with sterile water or saline inside the patient's bladder in order to keep the catheter in place. Thus, fluid from the bladder exits through the hollow passageway of the catheter to the exterior of the patient's body. Examples of such prior art devices and procedures are disclosed in U.S. Publication No. U.S. 2003/0009079, U.S. Pat. No. 4,867,745 and U.S. Pat. No. 4,143,651.

U.S. Pat. No. 4,888,000 discloses a supra pubic catheter inserted by way of a cannula which is positioned using a trocar, the trocar being withdrawn to leave the cannula in position through the wall of the bladder, allowing passage of the supra pubic catheter, the cannula being provided with means for subsequent removal from the catheter.

U.S. Pat. No. 5,520,636 teaches a device for flushing the urinary bladder.

U.S. Pat. No. 6,350,255 illustrates a pad for use with a continent ostomy port which includes a body portion having a internal wall defining an aperture appropriately sized placed around a stoma, the body portion of the pad being sized and shaped for placement against the user's skin beneath a face place of an ostomy port. The pad is formed of a soft, flexible material to thereby cushion and protect the skin from contact with the ostomy port face plate.

One of the difficulties with the prior art devices is that the inflated balloon does not always stay in place, either due to the patient's movement, movement of the catheter, or the weight of the saline filled balloon. When a good seal is not maintained, urinary leakage can occur around the balloon. The patient becomes wet, and in addition the weight of the patient in bed and the pressure on the skin increases a patient's risk of infection, skin breakdown and formation of decubitus ulcers. There is a general concern for decubitus ulcers in the sacrum and bilateral hips which manifest itself as Ishial tuberosities. If a patient already has decubitus ulcers in these areas with urine leakage on such dressing affects the effectiveness of the dressing and the medication under the dressing, which ultimately leads to various complications.

Moreover, leakage of air and fluid around catheter provides an avenue for infection. If air and or fluid can leak out, organisms can be introduced through this access. The catheter outside the skin and any organisms on this catheter will be deposited in the bladder by the migration and play of the catheter.

Moreover, it has been documented that, without properly securing the urethral catheter, the stoma over time, becomes larger and eventually causing insertion of a larger catheter. This causes the patient more trauma and discomfort. Much of the existing difficulties may stem from the fact that urethral catheters are used in supra pubic catheter applications.

Retention bolsters for gastrostomy and other ostomy tubes have been disclosed as illustrated in the U.S. Pat. No. 6,039,714. Moreover, retention bolster for percutaneous catheters which have a convexly curved exterior surface which contacts the epidermal surface of the patient are shown on U.S. Pat. No. 5,484,420.

Finally, U.S. Pat. No. 3,976,080 teaches an endotracheal tube holder.

Accordingly, there is a need for improved apparatus and method as well as system for a supra public catheter.

### SUMMARY OF THE INVENTION

The present invention teaches certain benefits in construction and use which give rise to the objectives described below.

The present invention provides a retention element for securing a catheter having an inflatable balloon within a stomal tunnel of a patient such that the inflatable balloon seals against the stomal tunnel. The retention element comprises an axial portion having an internal conduit adapted to receive the catheter therethrough. The axial portion is adapted to engage the catheter and resist movement of the catheter with respect to the retention element. The retention element further includes a radially extending flange portion having an annular sidewall extending outwardly from the axial portion to an outer perimeter. The radially extending flange portion is adapted to contact the patient and secure the catheter, thereby retaining the inflatable balloon in a position to seal the stomal tunnel. In the preferred embodiment, the radially extending flange portion is generally conical in shape, such that only the outer perimeter of the radially extending flange portion contacts the patient at a contact location that is a distance from the stomal tunnel, and the annular sidewall does not contact the patient adjacent the stomal tunnel.

A primary objective of the present invention is to provide a retention element having advantages not taught by the prior art.

Another objective is to provide a retention element that holds the catheter is a proper position so that the inflatable balloon seals the stomal tunnel and does not allow leakage.

Another objective is to provide a retention element that does not contact, compress, or otherwise damage the tissue adjacent to or around the stomal tunnel.

Another objective is to provide a retention element that enables air to circulate adjacent the stomal tunnel.

A further objective is to provide a retention element that is transparent, thereby allowing a user to promptly visualize tissue damage adjacent the stomal tunnel at the earliest stage of damage, before the tissue damage becomes more advanced and severe.

Other features and advantages of the present invention will become apparent from the following more detailed description, taken in conjunction with the accompanying drawings, which illustrate, by way of example, the principles of the invention.

### BRIEF DESCRIPTION OF THE DRAWING

The accompanying drawings illustrate the present invention. In such drawings:

FIG. 1 is a side elevational view of a catheter system that includes a catheter and a retention element;

FIG. 2 is a perspective view of the retention element;

FIG. 3 is a side elevational sectional view of the catheter system;

FIG. 4 is a side elevational view of a second embodiment of the catheter system, illustrating a second embodiment of the retention element;

FIG. 5 is a perspective view of the second embodiment of the retention element;

FIG. 6 is a side elevational sectional view thereof;

FIG. 7 is a perspective view of a third embodiment of the retention element;

FIG. 8 is a top plan view thereof;

FIG. 9A is a perspective view of a fourth embodiment of the retention element, illustrating the retention element in an open configuration;

FIG. 9B is a perspective view thereof illustrating the retention element in a closed configuration;

FIG. 10 is a perspective view of a fifth embodiment of the retention element;

FIG. 11A is a perspective view of a sixth embodiment of the retention element;

FIG. 11B is a top plan view thereof;

FIG. 11C is an enlarged view of a portion of the retention element;

FIG. 12 is a perspective view of a seventh embodiment of the retention element; and

FIG. 13 is a side elevational view of a prior art catheter system that does not include the retention element.

### DETAILED DESCRIPTION OF THE INVENTION

The above-described drawing figures illustrate the invention, a catheter system 10 that includes a retention element 30 for securing a catheter 12 having an inflatable balloon 18 within a stomal tunnel 20 of a patient 24 such that the inflatable balloon 18 seals against the stomal tunnel 20.

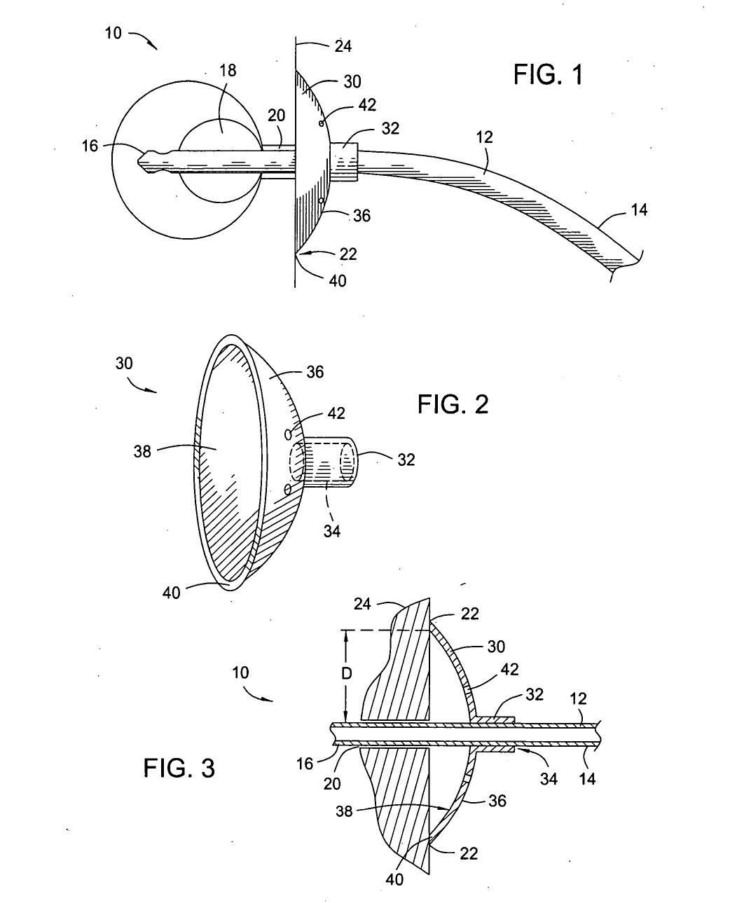

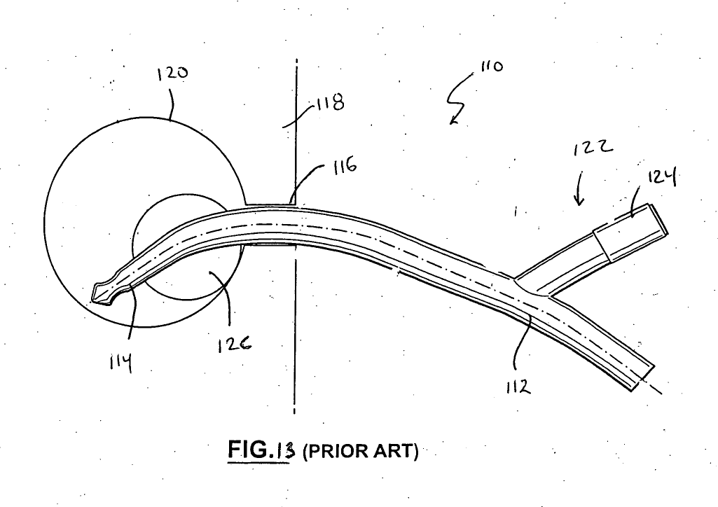

As shown in FIG. 1, the catheter 12 is similar to a prior art catheter 110 illustrated in FIG. 13. The catheter 12 includes a proximal end 14 and a distal end 16 having an inflatable balloon 18. The inflatable balloon 18 is adapted to be inflated to abut and seal the stomal tunnel 20, as is well known in the art. While we discuss the particular situation of using the catheter 12 for draining a bladder, the retention element 30 of the catheter system 10 could also be adapted for use with gastrostomy tubes and other similar tubes or catheters, and the terminology used in the present application is specifically defined to include use of the retention element 30 and the catheter system 10 for other similar or equivalent medical uses.

As shown in FIGS. 1-3, the retention element 30 comprises an axial portion 32 having an internal conduit 34 adapted to receive the catheter 12 therethrough. The axial portion 32 is adapted to engage the catheter 12 and resist movement of the catheter 12 with respect to the retention element 30. In the embodiment of FIGS. 1-3, the internal conduit 34 frictionally engages the catheter 12. In alternative embodiments, the internal conduit 34 may be internally threaded, or may include a form of locking post or element, described below, or any similar or equivalent element that may be devised by those skilled in the art.

The retention element 30 further includes a radially extending flange portion 36 having an annular sidewall 38 extending outwardly from the axial portion 32 to an outer perimeter 40. The radially extending flange portion 36 is preferably generally conical in shape, such that only the outer perimeter 40 of the radially extending flange portion 36 is adapted to contact a patient 24 at a contact location 22 that is a distance D from the sensitive, often vulnerable tissue at the entrance of a stomal tunnel 20, and such that the annular sidewall 38 does not contact the patient 24 at the sensitive site adjacent the stomal tunnel 20. For purposes of this application, the term “generally conical” shall be broadly defined to include alternative shapes that maintain the functional relationship wherein the outer perimeter 40 functionally contacts the patient 24 at the contact location 22, spaced from the stomal tunnel 20, without contacting and irritating the opening of the stomal tunnel 20.

In the preferred embodiment, the radially extending flange portion 36 is transparent, thereby enabling the user or the clinician to readily spot any tissue damage around the stomal tunnel 20. The radially extending flange portion 36 is preferably resilient, most preferably being constructed of resilient plastic. The resilient nature of the radially extending flange portion 36 ensures that the radially extending flange portion 36 will not exert enough pressure on the patient 24 to damage tissue adjacent the stomal tunnel 20, but will flex and thereby relieve the pressure on the contact location 22 of the patient 24.

The radially extending flange portion 36 preferably also includes at least one aperture 42 therethrough, so that air can circulate to the area adjacent the stomal tunnel 20.

The retention element 30 enables a unique method for securing a catheter 12 in a patient 24 having a stomal tunnel 20. The method comprising the steps of first providing the catheter 12 and the retention element 30 described above. The catheter 12 is inserted into the stomal tunnel 20 of a patient 24 such that the inflatable balloon 18 seals against the stomal tunnel 20. As is well known in the prior art, the inflatable balloon 18 is inserted in a deflated condition (not shown), and then inflated with a fluid. Since the general structure and use of the catheter 12 and inflatable balloon 18 are well known, they are not described in greater detail herein.

Once the catheter 12 has been properly positioned in the patient, the retention element 30 is used to lock the catheter 12 in place. The catheter 12 is positioned through the axial portion 32 of the retention element 30 such that the retention element 30 engages the catheter 12 and resists movement of the catheter 12 with respect to the retention element 30. The retention element 30 may be positioned on the catheter 12 before or after the catheter 12 has been inserted into the patient 24, either slidably engaged onto the catheter 12 from a distal end 16 before the inflatable balloon 18 is inflated, or wrapped around the catheter 12, as described below.

The retention element 30 is then moved up the catheter 12 towards the patient 24 until the outer perimeter 40 of the radially extending flange portion 36 contacts the patient 24 at a contact location 22 that is a distance D from a stomal tunnel 20, and the annular sidewall 38 does not contact the patient adjacent the stomal tunnel 20, thereby retaining the inflatable balloon 18 in a position to seal the stomal tunnel 20.

By constructing the retention element 30 such that only the outer perimeter 40 contacts the patient 24, such that the contact location 22 is spaced a distance D from the stomal tunnel 20, it is ensured that the tissue of and adjacent to the stomal tunnel 20 is not damaged by the retention element 30.

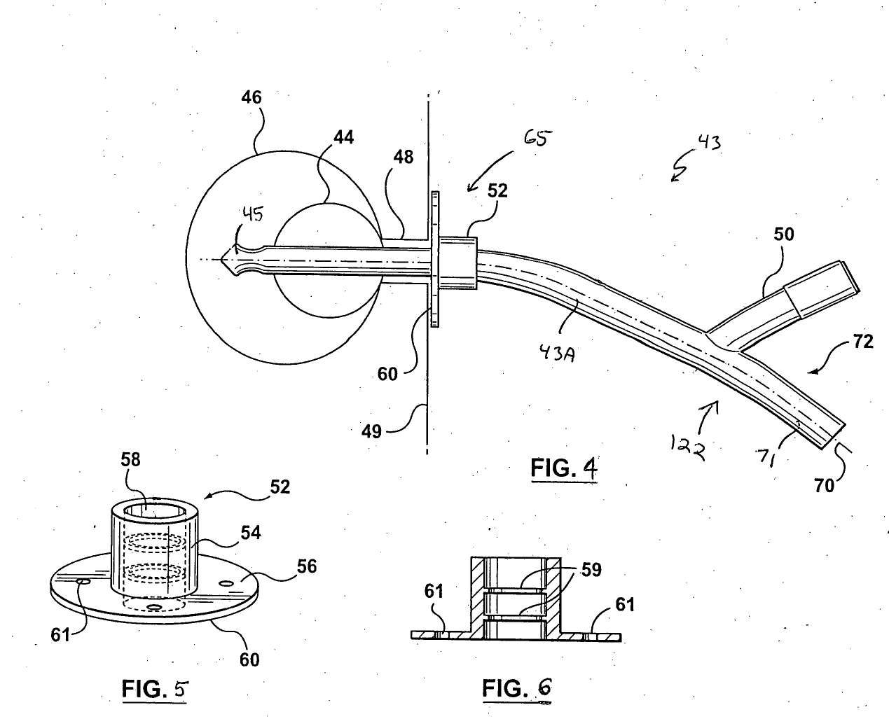

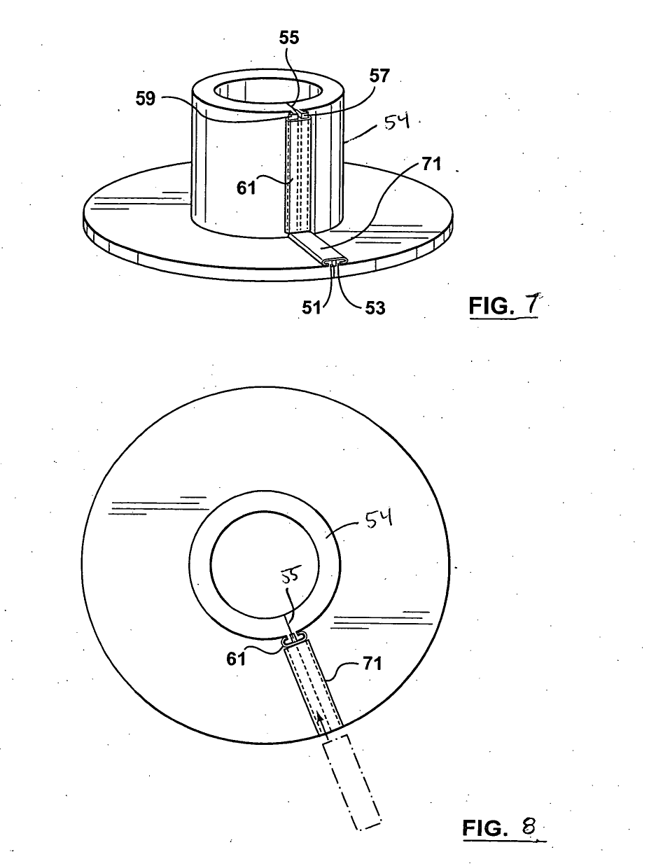

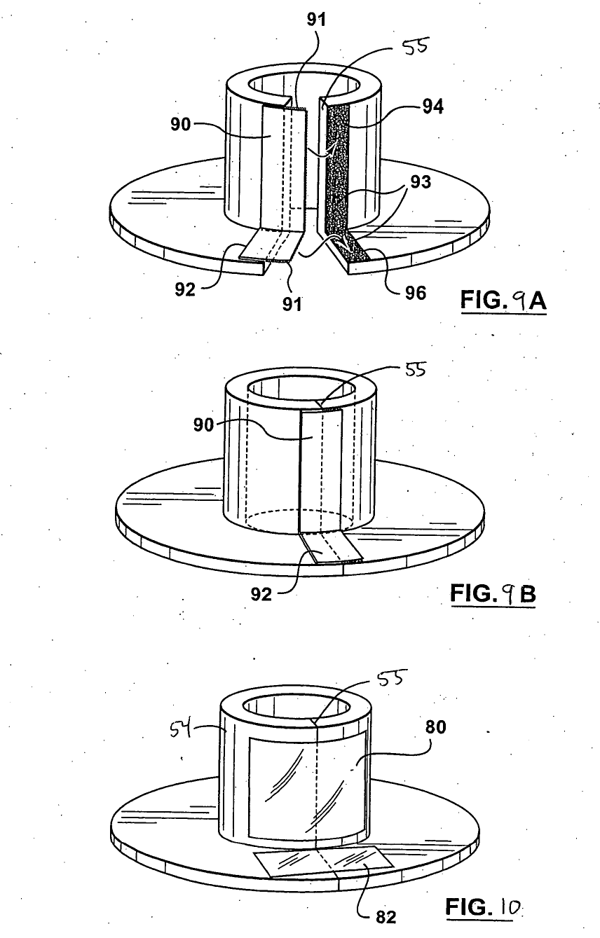

FIG. 4 is a side elevational view of a second embodiment of the catheter system 43, illustrating a second embodiment of the retention element 65. FIG. 5 is a perspective view of the second embodiment of the retention element 65, and FIG. 6 is a side elevational sectional view thereof. As shown in FIG. 4, the second embodiment of the catheter 43A has at its distal end 45 an inflated balloon 44. In particular, the catheter 43A is inserted directly into the bladder 46 through the supra pubic area through a stomal tunnel 48 in the abdomen or pelvic wall. The balloon 44 on the catheter 43A is then inflated within the bladder 46 by means of one of the branches 50 of a “Y” branch 122, which is adapted for inserting saline solution, in a manner well known to those persons skilled in the art. Upon inflating the balloon 44, the catheter 43A is then pulled snuggly to occlude the opening from the bladder 46 to the stomal tunnel 48. FIG. 4 also discloses that one of the branches 71 communicates with the remainder of the catheter 43 to present a passage way which permits voiding of urine through the catheter 43.

The second embodiment of the retention element 65, illustrated in FIGS. 5 and 6, includes a flange 52 that is adapted to be disposed along the catheter 43A. The flange 52 consists of a sleeve and hollow cylindrical portion 54 having a hole or bore 58 there through. Cylindrical portion 54 is symmetrical about axis 60. The retainer device 52 also comprises the radially extending flange portion 56. In particular, the inner diameter of the hole 58 is sized so as to be slidably moveable along the longitunal axis 70 of catheter 43 in the region between the pelvic wall 49 and the proximal end 72 of the catheter 43, yet frictionally engage the outer diameter of catheter 43 when contacting the epidermal skin layer of the patient.

In one embodiment, the hole 58 further includes a capturing means 59 disposed on the interior of the hole 58. In particular the capturing means 59 comprise a plurality of internally disposed projections or rings 59. Therefore the catheter 43 which is comprised of stretchable material is adapted to be received co axially within hole 58 and ride past the rings 59. Once the retaining device is placed in the desired position, the flexible nature of the retainer and catheter relaxes, the rings 59 assist in retaining the retainer 52 in the desired position on the catheter.

In the embodiment shown in FIGS. 4-6, the retainer 52 also includes one wall 60 which is adapted to contact the pelvic wall 49. The catheter 43 is inserted directly into the bladder 46 through the supra pubic area through stomal tunnel 48. The balloon 44 on the catheter 43 is inflated within the bladder 46 and pulled snuggly to occlude the opening from the bladder 46 to the stomal tunnel 48. The retainer 52 is then slidably disposed along the axial length 70 (exterior of the body of the patient) towards the balloon 44 so as to secure the balloon 44 under slight tension in its ideal location thereby creating a substantially closed or sealed system. In other words the length of catheter between the balloon 44 and the disc 52 contacting the exterior skin is under slight tension.

The system described herein comprises: (a) Placing a supra pubic catheter through a stomal tunnel from the outside skin of the abdomen of the patient to the bladder; (b) Inflating a balloon disposed at one end of the catheter, in said bladder to maintain said one end of the catheter in said bladder; and (c) Moving a retainer along the catheter towards the abdomen to draw said catheter balloon against the bladder. With the system described herein, the retainer holds the balloon over the inner, namely the bladder side opening to the stomal tunnel, maintaining the balloon in place and allowing the balloon to seat. It is important that the balloon remain seated so that the mucosa in the tunnel can dry and crust, thus permitting healing to take place. Thereby, the opportunity for infection is minimized thus improving the patient comfort and providing the least disruption to the body image.

The system described herein provides the following advantages: Reduces leakage from the stoma opening, due to a seal created between the flange and balloon. The catheter stays in place, as the flange secures the catheter from any type of movement within the stoma. The skin breakdown is significantly reduced, because the leakage is stopped since the stoma is sealed by the balloon secured in place by the retaining device. Infection to the stoma area is reduced when the seal is created, the seal functioning to inhibit any foreign matter or organisms from entering the stoma tunnel and the bladder, thereby lowering the incident of Urosepsis and sepsis. With a seal, the stoma has a chance to heal; since air contacts the normal mucosa in the stomal tunnel thus allowing granulation and healing. Stoma enlargement is minimized by eliminating migration, movement, and friction, which enlarges the stoma tunnel. Patient comfort and body image is significantly improved, because the seal that takes place keeps the area from leaking, giving the patient peace of mind that they will not have leakage in public. Physical activity of the patient is significantly improved since the seal keeps the area from leaking which intern allows the patient the freedom to participate in activities they normally would not with previously used catheters. Moreover, the catheter can be comprised of plastic such as bio compatible polymers, including silicone, rubber, silicone elastomers, polyurethane, silicone copolymers, polypropylene and/or similar materials or combination thereof typically used in the art. The flange can be comprised of the similar material. Furthermore, the invention comprises the kit which includes the supra pubic catheter and retainer, or just the retainer which can be used with a prior art catheter. Moreover other embodiments of the flange may be used to frictionally engage the catheter in the operable position described above and releasable from the catheter in a second inoperable position.

FIG. 7 is a perspective view of a third embodiment of the retention element, and FIG. 8 is a top plan view thereof. As illustrated in FIGS. 7-8, the retention element includes the flange having the cylindrical portion 54 cut axially and the flange portion 56 cut radially as shown, so that the flange can be opened as shown in FIG. 8 and closed as shown in FIG. 7. Generally speaking the flange material comprises of a flexible material such as plastic including biocompatible polymers such as silicon, rubber, silicon elastomers, polyurethane silicon copolymers, polypropylene and/or similar materials or combinations thereof typically used in the art. Such materials have a memory so that the flange as shown in FIG. 7 would tend to move to a closed position, as shown in FIG. 8.

Accordingly, the flange shown in FIG. 7 could be manipulated so that the cut edges 57 and 59 of the cylindrical portion and the cut edges 61 and 63 of the flange portion 56 could be spread apart so as to receive the catheter 43 as shown in FIG. 8.

FIG. 9A is a perspective view of a fourth embodiment of the retention element, illustrating the retention element in an open configuration. FIG. 9B is a perspective view thereof illustrating the retention element in a closed configuration. As shown in FIGS. 9A and 9B, the retention element can be equipped with hook and loop material, such as VELCRO®. In particular, the hook and loop material 90 can be used to secure the flange around the catheter as previously discussed. Alternatively, the hook and loop material shown in FIG. 9B can be pulled apart to the position shown in FIG. 9A. Furthermore, the hook and loop materials 94 and 96 can extend further along the cylindrical portion 54 as well as the flange portion 56 so that the edge 57 can extend beyond the edge 59 so as to make a smaller diameter bore 58. In this manner, the edge of flange 61 would also extend beyond edge 63.

FIG. 10 is a perspective view of a fifth embodiment of the retention element. As shown in FIG. 10, adhesive tape 80 and 82 can be utilized so as to secure the flange frictionally along the catheter as previously described.

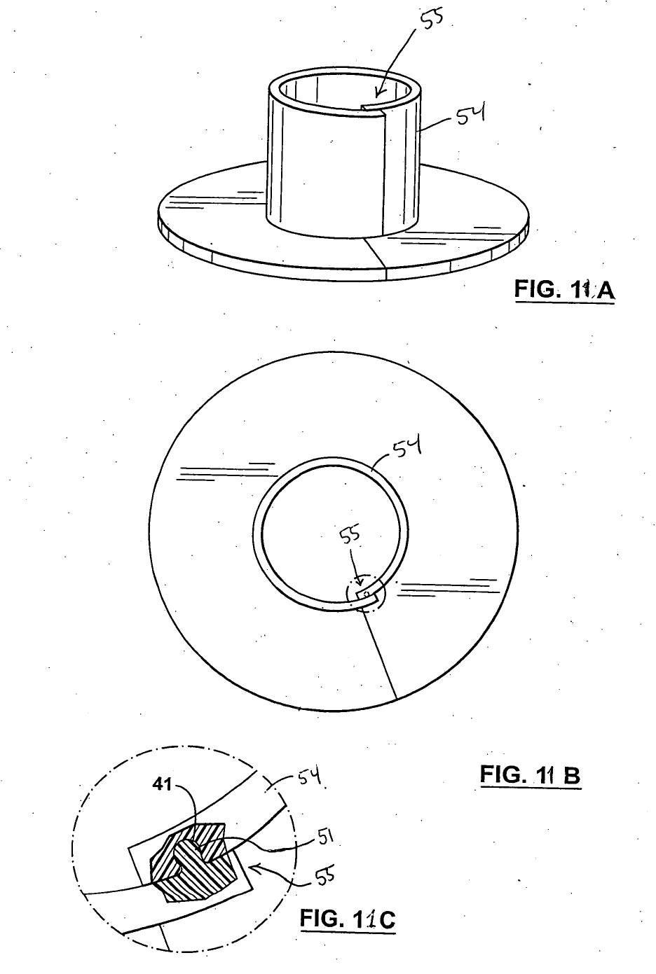

FIG. 11A is a perspective view of a sixth embodiment of the retention element. FIG. 11B is a top plan view thereof, and FIG. 11C is an enlarged view of a portion of the retention element. FIGS. 11A, 11B, and 11C illustrate another embodiment of the invention whereby the cylindrical portion 54 includes a recess 41 to releasably receive a projection 51, as shown.

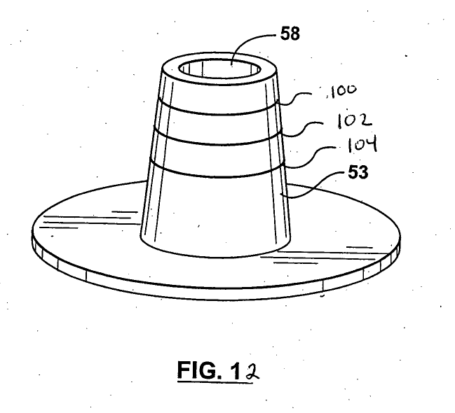

FIG. 12 is a perspective view of a seventh embodiment of the retention element wherein the retention element has a tapered cylindrical portion 53 with indicia or marks 100, 102, and 104, which correspond to different sized outer diameter of catheters. Accordingly, the friction fit between the inner bore 58 of the flange can be selected by cutting along the mark 100, 102, and 104 for the appropriate sized diameter to frictionally fit with the catheters previously described. For example markation line 100 may indicate a three millimeter diameter catheter while markation line 102 can indicate a 5 millimeter diameter catheter and markation line 104 indicate a 7 millimeter outer diameter catheter. Thereafter, the outer diameter of the cylindrical portion of the flange can be cut along one of the lines so as to present a flange having an appropriately sized inner diameter to frictionally fit with the outer diameter of the flange. Obviously, alternative measurements may be devised by one skilled in the art.

FIG. 13 illustrates a prior art urethral or urinary catheter 110 which has a proximal end 112 and a distal end 114. The proximal end 112 of the catheter 110 includes a “Y” branch 122 where one of the branches 124 is adapted for inserting saline solution through the catheter 110 so as to inflate a balloon 126 in a manner well known to those persons skilled in the art.

The distal end 114 is adapted to be placed through a stoma tunnel 116 of a body 118 towards a bladder 120. The stomal tunnel 116 is produced by a trocar under sterile conditions, using techniques that are known in the art. Once the balloon 126 is inflated, the balloon 126 functions to seal the stoma tunnel 116.

The terminology includes the words described above, similar or equivalent words, and derivatives thereof. Additionally, the words “a,” “an,” and “one” are defined to include one or more of the referenced item unless specifically stated otherwise. Also, the terms “have,” “include,” “contain,” and similar terms are defined to mean “comprising” unless specifically stated otherwise.

While the invention has been described with reference to at least one preferred embodiment, it is to be clearly understood by those skilled in the art that the invention is not limited thereto. Rather, the scope of the invention is to be interpreted only in conjunction with the appended claims.

What is claimed is:

1. A retention element for securing a catheter having an inflatable balloon within a stomal tunnel of a patient such that the inflatable balloon seals against the stomal tunnel, the retention element comprising:
an axial portion having an internal conduit adapted to receive the catheter therethrough, the axial portion being adapted to engage the catheter and resist movement of the catheter with respect to the retention element; and
a radially extending flange portion having an annular sidewall extending outwardly from the axial portion to an outer perimeter,
wherein the radially extending flange portion contacts the patient and secures the catheter, thereby retaining the inflatable balloon in a position to seal the stomal tunnel, and wherein the radially extending flange portion is generally conical in shape.

2. The retention element of claim 1, wherein the radially extending flange portion is shaped such that only the outer perimeter of the radially extending flange portion is adapted to contact the patient at a contact location that is a distance from the stomal tunnel, and the annular sidewall does not contact the patient adjacent the stomal tunnel

3. The retention element of claim 2, wherein the radially extending flange portion is transparent.

4. The retention element of claim 2, wherein the radially extending flange portion is resilient.

5. The retention element of claim 4, wherein the radially extending flange portion will not exert enough pressure on the patient to damage tissue adjacent the stomal tunnel, but will flex and thereby relieve the pressure on the contact location of the patient.

6. The retention element of claim 2, wherein the radially extending flange portion includes at least one aperture therethrough.

7. A catheter system comprising:
a catheter having an inflatable balloon;
a retention element comprising an axial portion having an internal conduit adapted to receive the catheter therethrough, the axial portion being adapted to engage the catheter and resist movement of the catheter with respect to the retention element; and
a radially extending flange portion of the retention element having an annular sidewall extending outwardly from the axial portion to an outer perimeter,
wherein the radially extending flange portion is generally conical in shape, such that only the outer perimeter of the radially extending flange portion is adapted to contact a patient at a contact location that is a distance from a stomal tunnel, and the annular sidewall does not contact the patient adjacent the stomal tunnel.

8. The catheter system of claim 7, wherein the radially extending flange portion is transparent.

9. The retention element of claim 7, wherein the radially extending flange portion is resilient.

10. The catheter system of claim 9, wherein the radially extending flange portion will not exert enough pressure on the patient to damage tissue adjacent the stomal tunnel, but will flex and thereby relieve the pressure on the contact location of the patient.

11. The catheter system of claim 7, wherein the radially extending flange portion includes at least one aperture therethrough.

12. A method for securing a catheter in a patient having a stomal tunnel, the catheter having an inflatable balloon, the method comprising the steps of:
providing a retention element comprising an axial portion having an internal conduit, and a radially extending flange portion of the retention element having an annular sidewall extending outwardly from the axial portion to an outer perimeter, wherein the radially extending flange portion is generally conical in shape;
inserting a catheter within a stomal tunnel of a patient such that the inflatable balloon seals against the stomal tunnel;
positioning the catheter through the axial portion of the retention element such that the retention element engages the catheter and resists movement of the catheter with respect to the retention element the retention element; and
moving the retention element up the catheter towards the patient until the outer perimeter of the radially extending flange portion is contacts the patient at a contact location that is a distance from a stomal tunnel, and the annular sidewall does not contact the patient adjacent the stomal tunnel, thereby retaining the inflatable balloon in a position to seal the stomal tunnel.
